(19) United States
(12) Patent Application Publication
(10) Pub. No.: US 2022/0338816 A1
(43) Pub. Date: Oct. 27, 2022

(54) FULLY AUTOMATED CARDIAC FUNCTION AND MYOCARDIUM STRAIN ANALYSES USING DEEP LEARNING

(71) Applicant: Shanghai United Imaging Intelligence Co., LTD., Shanghai (CN)

(72) Inventors: Xiao Chen, Cambridge, MA (US); Abhishek Sharma, Cambridge, MA (US); Terrence Chen, Cambridge, MA (US); Shanhui Sun, Cambridge, MA (US)

(73) Assignee: Shanghai United Imaging Intelligence Co., LTD., Shanghai (CN)

(21) Appl. No.: 17/236,173

(22) Filed: Apr. 21, 2021

(51) Int. Cl. A61B 5/00; G06N 3/04; G06N 3/08; G16H 30/40; G16H 50/30

(52) U.S. Cl. CPC A61B 5/7267; G06N 3/04; G06N 3/08; G16H 30/40; G16H 50/30; A61B 5/0044; A61B 2576/023

(57) ABSTRACT

A system and method for cardiac function and myocardial strain analysis include techniques and structure for classifying a set of cardiac images according to their views, detecting a heart range and valid short-axis slices in the set of cardiac images, determining heart segment locations, segmenting heart anatomies for each time frame and each slice, calculating volume related parameters, determining key physiological time points, calculating myocardium transmural thickness and deriving a cardiac function measure from the myocardium transmural thickness at the key physiological time points, estimating a dense motion field from the key physiological time points as applied to the set of cardiac images, calculating myocardial strain along different myocardium directions from the dense motion field, and providing the cardiac function measure and myocardial strain calculation to a user through a user interface.

### BACKGROUND

The aspects of the present disclosure relate generally to the study of cardiac physiology, and in particular to automating the analysis of cardiac function and myocardial strain.

Cardiac function and myocardium strain analyses are crucial for the diagnosis and treatment of cardiovascular disease. Cardiac function analyses generally include heart chamber volume measurement, ejection fraction, and myocardium thickness among others. Myocardium strain measures myocardial deformation from an estimation of myocardium motion and has been demonstrated to be a comprehensive, sensitive and early indicator of cardiac dysfunction. These analyses are complicated and require extensive domain expertise. Through the years tremendous efforts have been made to simplify and automate the various processes involved in cardiac function analysis and myocardial strain analysis. However, current solutions still require a large amount of human observation, manipulation of the views, and interpretation of the results.

Human intervention in these processes, which usually requires extensive training and experience, may generally lead to issues of inter- and intra-observer variability, may provide inferior reproducibility of the analyses, and requires additional time and effort.

### SUMMARY

It would be advantageous to provide a method and system that automates analyses of cardiac function and myocardial strain.

According to an aspect of the present disclosure, a method includes classifying a set of cardiac images according to their views; detecting a heart range and valid short-axis slices in the set of cardiac images; determining heart segment locations; segmenting heart anatomies for each time frame and each slice; calculating volume related parameters; determining key physiological time points; calculating myocardium transmural thickness and deriving a cardiac function measure from the myocardium transmural thickness at the key physiological time points; estimating a dense motion field from the key physiological time points as applied to the set of cardiac images; calculating myocardial strain along different myocardium directions from the dense motion field; and providing the cardiac function measure and the myocardial strain to a user through a user interface.

The views may include short-axis, 2-chamber, 3 chamber, 4 chamber views.

The method may include detecting the heart range and valid short-axis slices in the set of cardiac images by detecting cardiac anatomical landmarks in the views.

The cardiac anatomical landmarks may comprise a mitral annulus and apical tip of a left ventricle.

Determining heart segment locations may include determining locations of a basal anterior, basal anteroseptal, basal inferoseptal, basal inferior, basal inferolateral, basal anterolateral, mid anterior, mid anteroseptal, mid inferoseptal, mid inferior, mid inferolateral, mid anterolateral, apical anterior, apical septal, apical inferior, apical lateral and apex of a left ventricle. Segmenting heart anatomies may include segmenting one or more of a left ventricle myocardium, right ventricle myocardium, left atrium blood pool, right atrium blood pool, papillary muscle, trabecular muscle, left ventricle blood pool and right ventricle blood pool.

According to another aspect of the present disclosure, a system includes a source of cardiac images, one or more neural networks configured to classify a set of cardiac images according to their views, detect a heart range and valid short-axis slices in the set of cardiac images, determine heart segment locations, segment heart anatomies for each time frame and each slice, calculate volume related parameters, determine key physiological time points, calculate myocardium transmural thickness and deriving a cardiac function measure from the myocardium transmural thickness at the key physiological time points, estimate a dense motion field from the key physiological time points as applied to the set of cardiac images, and calculate myocardial strain along different myocardium directions from the dense motion field, wherein the system further includes a user interface to provide the cardiac function measure and the myocardial strain to a user.

These and other aspects, implementation forms, and advantages of the exemplary embodiments will become apparent from the embodiments described herein considered in conjunction with the accompanying drawings. It is to be understood, however, that the description and drawings are designed solely for purposes of illustration and not as a definition of the limits of the disclosed invention, for which reference should be made to the appended claims. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### BRIEF DESCRIPTION OF THE DRAWINGS

In the following detailed portion of the present disclosure, the invention will be explained in more detail with reference to the example embodiments shown in the drawings. These embodiments are non-limiting exemplary embodiments, in which like reference numerals represent similar structures throughout the several views of the drawings, wherein:

FIG. 1 illustrates a general overview of a workflow for performing the cardiac function analysis and myocardial strain analysis;

FIG. 2 illustrates a schematic block diagram of an exemplary system incorporating aspects of the disclosed embodiments;

FIG. 3 illustrates an exemplary architecture of a computing engine that may be used to implement the disclosed embodiments;

FIG. 4 depicts an exemplary simple neural network that may be utilized to implement the disclosed embodiments;

FIG. 5 shows a flow diagram of the cardiac function analysis and strain analysis workflow; and

FIG. 6 illustrates an exemplary 17 segment bull's eye display.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirits and scope of the present disclosure. Thus, the present disclosure is not limited to the embodiments shown, but to be accorded the widest scope consistent with the claims.

It will be understood that the term “system,” “unit,” “module,” and/or “block” used herein are one method to distinguish different components, elements, parts, section or assembly of different level in ascending order. However, the terms may be displaced by other expression if they may achieve the same purpose.

It will be understood that when a unit, module or block is referred to as being “on,” “connected to” or “coupled to” another unit, module, or block, it may be directly on, connected or coupled to the other unit, module, or block, or intervening unit, module, or block may be present, unless the context clearly indicates otherwise. As used herein, the term “and/or” includes any and all combinations of one or more of the associated listed items.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an Erasable Programmable Read Only Memory (EPROM). It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

The terminology used herein is for the purposes of describing particular examples and embodiments only, and is not intended to be limiting. As used herein, the singular forms “a,” “an,” and “the” may be intended to include the plural forms as well, unless the context clearly indicates otherwise. It will be further understood that the terms “include,” and/or “comprise,” when used in this disclosure, specify the presence of integers, devices, behaviors, stated features, steps, elements, operations, and/or components, but do not exclude the presence or addition of one or more other integers, devices, behaviors, features, steps, elements, operations, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The disclosed embodiments are directed to a system and method for providing a more automated workflow for one or more of cardiac function analysis and strain analysis by leveraging deep learning techniques. The system and method described herein are described with a focus on the left ventricle, which is by far the most clinically studied chamber of the heart. However, it should be understood that the disclosed system and method may also be applicable to the right ventricle, left atrium, and right atrium. The workflow and techniques advantageously require little or no human intervention.

Various operations of the system and method for cardiac function analysis and strain analysis are described in the context of utilizing a neural network, and it should be understood that individual neural networks may be utilized for various operations, different networks may be used for combinations of various operations, or a single neural network may be utilized for all the operations.

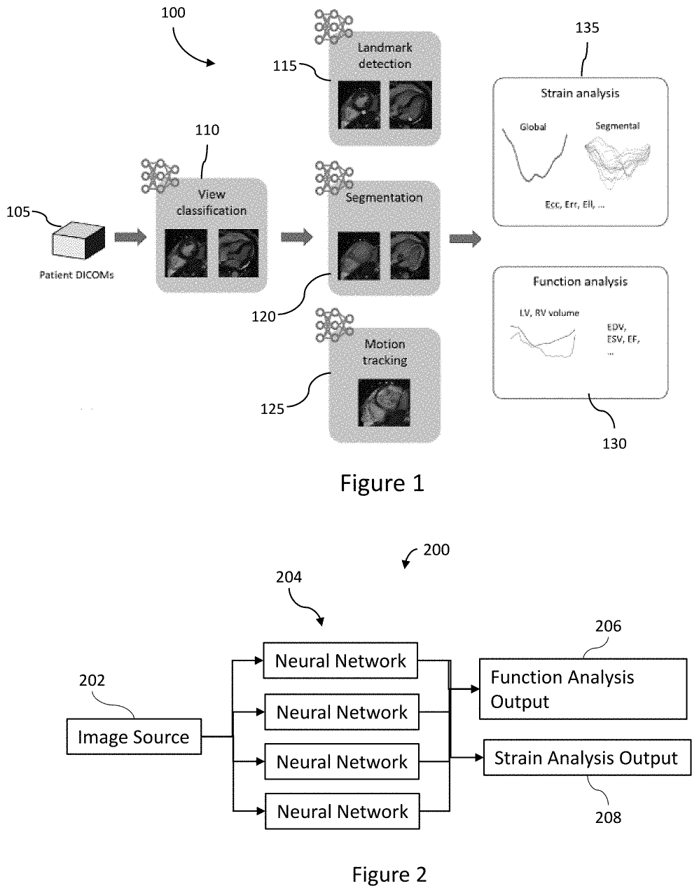

FIG. 1 illustrates a general overview of a workflow 100 for performing the cardiac function analysis and myocardial strain analysis. Cardiac images 105 may be classified according to their view 110, for example, using a neural network. The detection of cardiac landmarks 115 is used to select cardiac images that capture the range of the views and therefore may be used for analysis. Segmentation 120 may be performed on the selected images to identify sub-regions of the heart for analysis. Motion tracking 125 may then be used to analyze the cardiac function 130 and myocardial strain 135.

FIG. 2 illustrates a schematic block diagram of an exemplary system 200 incorporating aspects of the disclosed embodiments. The system 200 may include a source of cardiac images 202, for example, DICOM images, one or more neural networks 204 for performing the classification, landmark detection, and motion tracking functions, and one or more user interfaces, or other output devices 206, 208 for providing results of cardiac function analysis and myocardial strain analysis. It should be understood that the components of the system 200 may be implemented in hardware, software, or a combination of hardware and software.

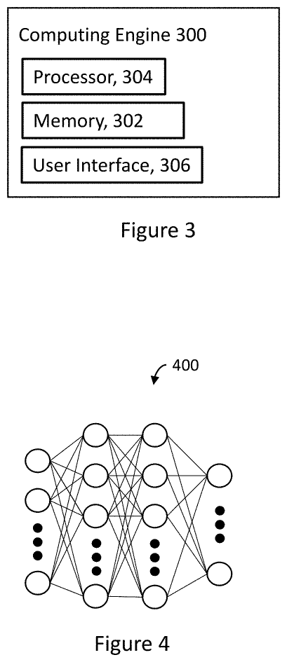

FIG. 3 illustrates an exemplary architecture of a computing engine 300 that may be used to implement the disclosed embodiments. The computing engine 300 may include computer readable program code stored on at least one computer readable medium 302 for carrying out and executing the process steps described herein. The computer readable program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The computer readable program code may execute entirely on the computing engine 300, partly on the computing engine 300, as a stand-alone software package, or partly or entirely on a remote computer or server, such as a cloud service.

The computer readable medium 302 may be a memory of the computing engine 300. In alternate aspects, the computer readable program code may be stored in a memory external to, or remote from, the computing engine 300. The memory may include magnetic media, semiconductor media, optical media, or any media which is readable and executable by a computer. The computing engine 300 may also include a computer processor 304 for executing the computer readable program code stored on the at least one computer readable medium 302. In at least one aspect, the computing engine 300 may include one or more input or output devices, generally referred to as a user interface 306 which may operate to allow input to the computing engine 300 or to provide output from the computing engine 300, respectively. The computing engine 300 may be implemented in hardware, software or a combination of hardware and software.

The computing engine 300 may generally operate to support one or more neural networks. FIG. 4 depicts an exemplary simple neural network 400 that may be utilized to implement the disclosed embodiments. While a simple neural network is shown, it should be understood that the disclosed embodiments may be implemented utilizing a deep learning model including one or more gated recurrent units (GRUs), long short term memory (LSTM) networks, fully convolutional neural network (FCN) models, generative adversarial networks (GANs), back propagation (BP) neural network models, radial basis function (RBF) neural network models, deep belief nets (DBN) neural network models, Elman neural network models, or any deep learning or machine learning model capable of performing the operations described herein. It should also be understood that the different functions of the disclosed embodiments, such as view classification, landmark detection, segmentation, and motion tracking may be implemented with individual neural networks, with a plurality of shared neural networks, or may be implemented with a single neural network.

The one or more neural networks 400 may be trained for the functions of view classification, landmark detection, segmentation, and motion tracking.

The one or more neural networks 400 may be trained with supervision to classify images into different views. Training data pairs of the view label and the image with or without the header information may be used. The image with or without the header information may be input to the one or more neural networks 400 and the one or more neural networks 400 may output a vector, with each element representing the probability the input information belongs to a certain category. The estimated probability vector may be compared to a ground truth label that has been converted to a one-hot vector. The difference can be measured using multi-class cross entropy and backpropagated to update parameters of the one or more neural networks 400 during training. The text information can be embedded into codes and concatenated with the input image or features in the one or more neural networks which may be a combination of convolutional layers and fully-connected layers.

The one or more neural networks 400 may be trained with supervision to detect landmarks in the image. In one approach, the landmark detection can be formulated as a segmentation task. Training data pairs may include the input image and the ground truth landmarks mask. The one or more neural networks 400 may take an input of the image and may output an image (mask) that only has targeted landmark(s) drawn. The estimation is compared to a ground truth landmark mask. For multiple landmarks, the landmark mask for each landmark can be put on different channels of the output. The difference can be measured using cross-entropy and backpropagated to update the neural network parameters during training. In some embodiments, the neural network may be a fully convolutional neural network.

The one or more neural networks 400 may also be trained with supervision to segment the heart anatomy. Training data pairs may include the input image and ground truth anatomy masks. Each pixel on the mask may have a value to represent the anatomy type to which it belongs. The one or more neural networks 400 may take an input of the image and may output an estimated segmentation mask. The estimation may be compared to ground truth. The difference may be measured using cross-entropy and may be backpropagated to update the neural network parameters during training. The one or more neural networks 400 may be one or more fully convolutional neural networks, for example, UNet.

The one or more neural networks 400 may further be trained with supervision to estimate the myocardium thickness. Training data pairs may include the input myocardium segmentation mask and a ground truth myocardium thickness map. The myocardium thickness map may have pixels with a non-zero value representing the myocardium thickness. The ground truth thickness map can be generated by calculating the equipotential surfaces in between the epicardium and endocardium and summing-up the distances across the surfaces. The one or more neural networks 400 may take an input of the myocardium mask and output the thickness map. The estimation may be compared to a ground truth. The difference can be measured using L2 loss and backpropagated to update the neural network parameters during training. The one or more neural networks 400 may be one or more fully convolutional neural networks, for example, UNet.

The one or more neural networks 400 may be trained in an unsupervised manner to estimate the motion in between two images. Training data may include two images where one is a reference image and the other is the moving image. The one or more neural networks 400 may take the two images and output a dense motion field representing the motion, which can be further used to warp the moving image. The warped moving image is compared to the reference image. The difference can be measured using L2 loss. By minimizing the difference, the warped image becomes similar to the reference image and leads to a more accurate estimation of the motion. This may be implemented using a fully convolutional neural network, for example, UNet.

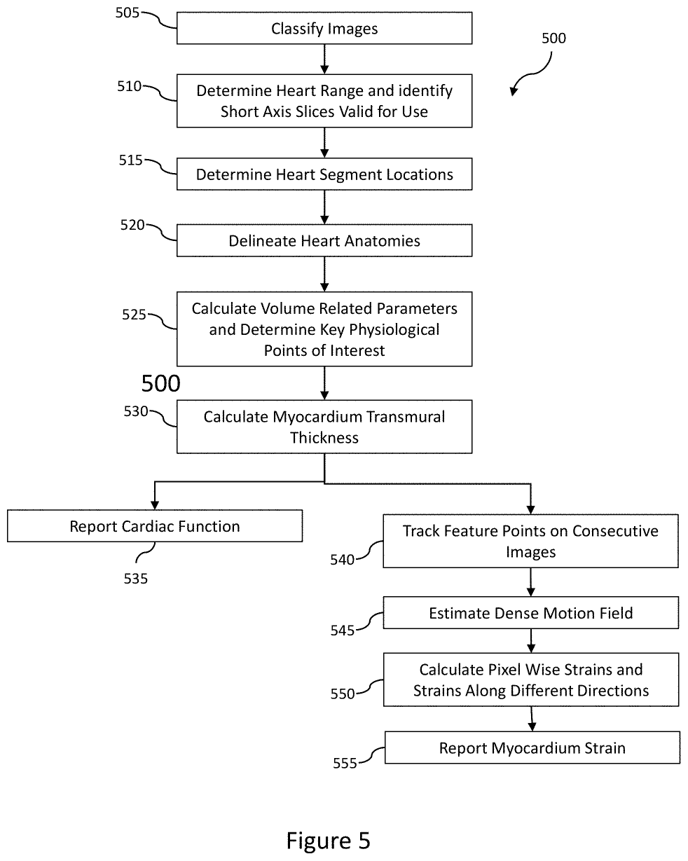

The disclosed workflow and techniques for implementing the workflow 500, shown in FIG. 5, generally utilize various images of the heart as described above, for example, scanned multi-slice DICOM images. The images are generally obtained as a series of slices at particular locations over the entire cardiac cycle.

As shown in block 505, the workflow for cardiac function analysis and strain analysis initially operates to utilize a neural network to classify the images according to various views, for example, short-axis, 2-chamber, 3-chamber, 4-chamber and other views as required. The classification operation is advantageous because different clinical parameters may be obtained from different views. The neural network generally operates to classify the images as short-axis, 2-chamber, 3-chamber, 4-chamber and other views as required by analyzing one or more of image header information and image content, for example, DICOM header information or DICOM image content.

Previous techniques generally require human intervention to visually classify the images, or to manually input the image header information, which may include for example, the scanning protocol, which may be prone to error. In the present embodiments, a neural network may be trained using both image header information and image content to automatically recognize and provide classification information of the images.

As shown in block 510, the workflow for cardiac function analysis and strain analysis may proceed to determine the heart range and to identify short axis slices that include the heart and are therefore valid for use in the analyses. For example, during image acquisition, some slices may be imaged out of the range of the heart, and are not useful for cardiac function or myocardial strain analysis. A neural network may be utilized to detect the heart range to find the valid short-axis slices that contains the heart. This can be achieved by detecting anatomical landmarks from the images. One example is to detect the range of left ventricle by detecting the mitral annulus and apical tip from the long-axis image. The left ventricle, which is by far the main studied chamber of the heart, is in between the mitral annulus and apical tip. Short-axis imaging planes that intersects the long-axis image in between the mitral annulus and the apical tip are considered to contain the left ventricle.

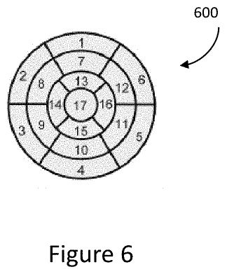

The workflow for cardiac function analysis and strain analysis may then proceed to determine heart segment locations as shown in block 515. A common standard for heart segmentation is the American Heart Association (AHA) 17 segment bull's eye display as shown in FIG. 6. The bull's eye display 600 generally segments the left ventricle into basal anterior 1, basal anteroseptal 2, basal inferoseptal 3, basal inferior 4, basal inferolateral 5, basal anterolateral 6, mid anterior 7, mid anteroseptal 8, mid inferoseptal 9, mid inferior 10, mid inferolateral 11, mid anterolateral 12, apical anterior 13, apical septal 14, apical inferior 15, apical lateral 16, and apex 17 segments. The one or more neural networks 400 may be one or more fully convolutional neural networks, for example, UNet, and may be used to determine from which bull's eye segment the image was derived. The one or more neural networks 400 may operate to detect the left ventricle range by detecting anatomical landmarks from the images, for example, the left ventricle-right ventricle insertion points, from which the ring-shape heart on the short-axis images can be divided into anterior, anteroseptal, inferoseptal, inferior, inferolateral and anterolateral regions. The heart may be divided into basal, mid and apical levels by dividing the space between the mitral annulus and the apical tip. The combination of the short-axis division and the long axis division results in the 16/17 AHA segments shown in FIG. 6.

As shown in block 525, the workflow for cardiac function analysis and strain analysis then includes delineating the heart anatomies. The delineation may be achieved by segmenting one or more of the left ventricle, right ventricle, left atrium, right atrium, and other anatomies such as the papillary muscles. A neural network may be used to perform the segmentation of these anatomies. The heart anatomy segmentation process generally includes segmenting one or more of a left ventricle myocardium, right ventricle myocardium, left atrium blood pool, right atrium blood pool, papillary muscle, trabecular muscle, left ventricle blood pool and right ventricle blood pool. It should be understood that any suitable heart anatomy may be included in the segmentation.

The heart anatomy segmentations may be performed on every slice obtained over the entire cardiac cycle.

The next process 530 in the workflow 500 includes calculating volume-related parameters and determining physiological related key time points of interest. From the heart anatomy segmentations, volume-related cardiac function parameters, for example, left ventricle chamber volume and myocardium mass, may be calculated by summing up the segmentation on each slice at each time frame and adjusted by the spatial resolution of the image voxel. The key physiological related time points, for example, the end-diastolic phase (ED) and end-systolic phase (ES) can then be determined from the left ventricle volume-time curve as the maximum and the minimum points on the curve, respectively. Routine clinical parameters, such as end-diastolic volume (EDV), ejection fraction (EF) may then be derived.

The workflow for cardiac function analysis and strain analysis 500 may then include calculating a myocardium transmural thickness 530 from the short-axis images or myocardium segmentation described above using a neural network. A left ventricle myocardium thickness may be defined on the whole left ventricle ring and thus regional and global values can be reported.

As shown in block 535, the cardiac function may then be reported using a bull's eye plot automatically derived from the determination of the heart segment locations described above.

As mentioned above, strain analysis requires an estimation of myocardium motion. A neural network may be used to track the feature points on consecutive images as shown in block 540 and estimate a dense motion field as shown in block 545. The myocardium region, defined by the segmentation mask on the ED frame, may be densely tracked through the entire cardiac cycle. As shown in block 550, pixel-wise strains may be calculated from the dense motion field, and strains along different directions such as longitudinal, circumferential and radial may be calculated from different views of the images. As shown in block 555, global and segmental strains can be reported by averaging over the whole heart and the 17 AHA segments 300 defined above. The strain values may be visualized in different formats such as the bullseye, curves and tables. The pixel-wise strains and motions may also be visualized as movies, along with the images.

The disclosed embodiments leverage deep learning techniques using one or more neural networks to provide a more automated workflow for one or more of cardiac function analysis and strain analysis. The use of one or more neural networks automates the workflow and provides consistency of the analyses in order to achieve faster and more accurate cardiac function assessment under various conditions with little or no human intervention.

Thus, while there have been shown, described and pointed out, fundamental novel features of the invention as applied to the exemplary embodiments thereof, it will be understood that various omissions, substitutions and changes in the form and details of devices and methods illustrated, and in their operation, may be made by those skilled in the art without departing from the spirit and scope of the presently disclosed invention. Further, it is expressly intended that all combinations of those elements, which perform substantially the same function in substantially the same way to achieve the same results, are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A method comprising:
classifying a set of cardiac images according to their views;
detecting a heart range and valid short-axis slices in the set of cardiac images;
determining heart segment locations;
segmenting heart anatomies for each time frame and each slice;
calculating volume related parameters;
determining key physiological time points;
calculating myocardium transmural thickness and deriving a cardiac function measure from the myocardium transmural thickness at the key physiological time points;
estimating a dense motion field from the key physiological time points as applied to the set of cardiac images;
calculating myocardial strain along different myocardium directions from the dense motion field; and
providing the cardiac function measure and the myocardial strain to a user through a user interface.

2. The method of claim 1, wherein the cardiac images are scanned multi-sliced DICOM images.

3. The method of claim 1, wherein the views comprise short-axis, 2-chamber, 3 chamber, 4 chamber views.

4. The method of claim 1, comprising detecting the heart range and valid short-axis slices in the set of cardiac images by detecting cardiac anatomical landmarks in the views.

5. The method of claim 4, wherein the cardiac anatomical landmarks comprise a mitral annulus and apical tip of a left ventricle.

6. The method of claim 1, wherein determining heart segment locations comprises determining locations of a basal anterior, basal anteroseptal, basal inferoseptal, basal inferior, basal inferolateral, basal anterolateral, mid anterior, mid anteroseptal, mid inferoseptal, mid inferior, mid inferolateral, mid anterolateral, apical anterior, apical septal, apical inferior, apical lateral and apex of a left ventricle.

7. The method of claim 1, wherein segmenting heart anatomies comprises segmenting one or more of a left ventricle myocardium, right ventricle myocardium, left atrium blood pool, right atrium blood pool, papillary muscle, trabecular muscle, left ventricle blood pool and right ventricle blood pool.

8. The method of claim 1, comprising using a neural network for classifying the set of cardiac images, detecting the heart range and valid short-axis slices, determining the heart segment locations, segmenting the heart anatomies, calculating the volume related parameters, determining the key physiological time points, calculating the myocardium transmural thickness, deriving the cardiac function measure, estimating the dense motion field, and calculating the myocardial strain.

9. The method of claim 8, wherein the neural network comprises one or more gated recurrent units, long short term memory networks, fully convolutional neural network models, generative adversarial networks, back propagation neural network models, radial basis function neural network models, deep belief nets neural network models, and Elman neural network models.

10. The method of claim 8, comprising training the neural network with supervision to classify the set of cardiac images, to detect cardiac anatomical landmarks in order to detect the heart range and valid short-axis slices, and to segment the heart anatomies.

11. The method of claim 8, comprising training the neural network without supervision to estimate motion between images to estimate the dense motion field.

12. A system comprising:
a source of cardiac images;
one or more neural networks configured to:
classify a set of cardiac images according to their views;
detect a heart range and valid short-axis slices in the set of cardiac images;
determine heart segment locations;
segment heart anatomies for each time frame and each slice;
calculate volume related parameters;
determine key physiological time points;
calculate myocardium transmural thickness and deriving a cardiac function measure from the myocardium transmural thickness at the key physiological time points;
estimate a dense motion field from the key physiological time points as applied to the set of cardiac images; and
calculate myocardial strain along different myocardium directions from the dense motion field; and
a user interface to provide the cardiac function measure and the myocardial strain to a user.

13. The system of claim 12, wherein the views comprise short-axis, 2-chamber, 3 chamber, 4 chamber views.

14. The system of claim 12, wherein the one or more neural networks are further configured to detect the heart range and valid short-axis slices in the set of cardiac images by detecting cardiac anatomical landmarks in the views.

15. The system of claim 12, wherein the, wherein the cardiac anatomical landmarks comprise a mitral annulus and apical tip of a left ventricle.

16. The system of claim 12, wherein the one or more neural networks are further configured to determine heart segment locations by determining locations of one or more of a basal anterior, basal anteroseptal, basal inferoseptal, basal inferior, basal inferolateral, basal anterolateral, mid anterior, mid anteroseptal, mid inferoseptal, mid inferior, mid inferolateral, mid anterolateral, apical anterior, apical septal, apical inferior, apical lateral and apex of a left ventricle.

17. The system of claim 12, wherein the one or more neural networks are further configured to segment one or more of a left ventricle myocardium, right ventricle myocardium, left atrium blood pool, right atrium blood pool, papillary muscle, trabecular muscle, left ventricle blood pool and right ventricle blood pool.

18. The system of claim 12, wherein the neural network comprises one or more gated recurrent units, long short term memory networks, fully convolutional neural network models, generative adversarial networks, back propagation neural network models, radial basis function neural network models, deep belief nets neural network models, and Elman neural network models.

19. The system of claim 12, wherein the neural network is trained with supervision to classify the set of cardiac images, to detect cardiac anatomical landmarks in order to detect the heart range and valid short-axis slices, and to segment the heart anatomies.

20. The system of claim 12, wherein the neural network is trained without supervision to estimate motion between images to estimate the dense motion field.
